(19) United States
(12) Patent Application Publication
(10) Pub. No.: US 2004/0175729 A1
(43) Pub. Date: Sep. 9, 2004

(54) Primer for nucleic acid amplification to detect carcinoembryonic antigen and test method using such primer

(75) Inventor: Kayo Hiyama, Kobe-shi (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATION

(21) Appl. No.: 10/702,770

(22) Filed: Nov. 7, 2003

(30) Foreign Application Priority Data
Nov. 8, 2002 (JP) 2002-324833

(51) Int. Cl. C12Q 1/68; C07H 21/04; C12P 19/34

(52) U.S. Cl. 435/6; 435/91.2; 536/24.3

(57) ABSTRACT

An object is to provide a new primer to use in genetic amplification reactions to detect carcinoembryonic antigen (CEA).

A primer for nucleic acid amplification is constructed by selecting from the region from No. 1900 to No. 2200 of the base sequence having SEQ ID No. 1 and its complementary strand, and by including oligonucleotides containing at least 5 or more continuous bases of SEQ ID No. 1 and/or its complementary strand, and oligonucleotides comprising a sequence selected from oligonucleotides or the complementary strand thereof comprising a base sequence having SEQ ID Nos. 2 to 24.

### FIELD OF THE INVENTION

The present invention relates to a primer for nucleic acid amplification in order to detect carcinoembryonic antigen.

### DESCRIPTION OF THE RELATED ART

Carcinoembryonic antigen (hereinafter referred to “CEA”) is a protein taken from human colon cancer, and is called carcinoembryonic antigen or carcinoembryonic protein because it shares common immunologic characteristics with embryonic tissue. CEA is the most useful tumor marker in clinical cancer. For example, related diseases include colon cancer, pancreatic cancer, bile duct cancer, lung cancer, stomach cancer, thyroid cancer, esophageal cancer, uterine cancer, malignant ovarian tumor, urinary tract cancer, etc. In colon cancer, higher CEA levels in the blood appear as the cancer progresses, and there are notable increases when metastasis to the liver occurs. Fifty to sixty percents of colon and pancreatic cancers indicate positive for CEA in the blood, but nearly all positive results indicate progressive cancer, and thus CEA levels are used in post-surgical and post-chemotherapy monitoring.

On the other hand, only a few percentage of sarcomas, leukemia and malignant lymphomas indicate positive, and CEA is negative in the majority of malignant tumors.

Oncocytes separate from their original lesion sites and metastasize throughout the body via blood circulation and the lymphatic system. In cancer surgery, the lesions must be removed as reliably as possible. Thus, it is necessary to accurately detect metastasis, and to conduct proper treatment corresponding to the extent of metastasis. For this reason, the intraoperative diagnosis of lymph node metastatic cancer is extremely important and highly significant. For example, in breast cancer, there is a trend to reduce the range of lymph node removal in order to improve the quality of life (QOL), and the intraoperative diagnosis of lymph node metastatic cancer can be an important indicator in order to decide the minimum range of lymph nodes to be removed. In esophageal cancer, detecting the sites of lymph node metastasis can be an indicator for deciding whether to select laparotomy, closed chest or cervical incision as the surgical approach. In prostate cancer, lymph node metastasis is an index for deciding whether to discontinue extractive surgery and conduct hormone therapy, etc. Similarly, lymph node metastasis can be an indicator in stomach cancer for selecting the surgical approach and the course of post-operative therapy. Considering the burden on the patient, the diagnosis of metastatic cancer during surgery must be reached quickly.

Many kits for the measurement of CEA are already commercially available (Manual of Laboratory Test Methods, Edition 31, pp. 674-675, KANEHARA & CO., LTD., Sep. 20, 1998).

The recent development of genetic analysis techniques has led to the effective diagnosis of cancer by detecting the expression of cancer marker genes. For example, the PCR method (Japanese Patent Provisional Publication No. 61-274697) can amplify the target DNA fragments several hundred thousand times by repeatedly denaturing the DNA strands to single strands, binding primers at both ends of a specific region in the DNA strands, and using DNA polymerase to synthesize DNA. This can be used as a highly sensitive method to analyze nucleic acids in a variety of samples. For example, it is useful in the diagnosis of infectious disease, genetic disease and cancer because the nucleic acids in samples derived from biological fluids and tissues of animal can be analyzed.

The RT-PCR method can be used to detect RNA. RT-PCR is a method of detection that extracts RNA from, for example, tumor tissue, uses an oligo (dT) or random hexamer primer to synthesize cDNA based on a reverse transcription (RT) enzyme, and amplifies the synthesized cDNA, using the PCR method. There were reports to use this method to diagnose fibroblastomas (Hokkaido Medical Journal, pp. 135-141, Vol. 66(2), (1991)). It is possible to detect mRNA from excised tissue using RT-PCR, to a certain extent, thereby it is possible to avoid the problem of overlooking metastatic cancer. This kind of nucleic acid amplification has become practical in the area of tumor and cancer diagnosis (Summary of Clinical Test Methods, Ed. 31, p. 1314, KANEHARA & CO., LTD., Sep. 20, 1998).

However, the PCR method needs the operation to denature the double stranded template DNA into single strands, and this amplification reaction must be conducted repeatedly under multiple temperature conditions. Generally, it takes about 2 hours in order to obtain a detectable amplified product, and this is not preferable for a intraoperative test that must be conducted rapidly.

Besides the PCR method, there have been reports on Loop-Mediated Isothermal Amplification (LAMP) method as a DNA amplification method (e.g., U.S. Pat. No. 6,410,278). DNA amplification in LAMP method has the potential to replace PCR because of its simplicity, rapidity, specificity and cost-effectiveness. The LAMP method is characterized in employing 4 different primers specifically designed to recognize 6 distinct regions on the target gene and its process being performed at a constant temperature using a strand displacement reaction.

The LAMP method amplifies genes by using multiple primers including a primer that forms a hairpin structure at the end of the amplification product when promoting the strand displacement reaction. First, in the initial reaction, a dumbbell structure having a single strand with loop parts at both ends is synthesized from the template DNA using 2 types of inner primers (FIP, RIP), 2 types of outside primers (F3 primer, R3 primer) and strand displacement DNA polymerase. This structure is the starting structure of the amplification cycle, and a DNA growth and synthesis reaction is promoted using this structure itself from the 3′ end side as the template. The amplification product comprises a multiple repeat structure, and the unit of the repeat structure comprises a complementary region within the same strand in which there are two inverted nucleic acid base sequences that comprise the region to be amplified between the primers.

The LAMP method does not require thermal control to denature the double stranded template DNA into single strands, and is characterized by continuous isothermic promotion (in the region of 65° C.) of all the amplification reactions (BIO Venture, Japan, 2001, Vol. 1, No. 1, pp. 109-115; BIO INDUSTRY, Japan, 2001, Vol. 18, No. 2, pp. 15-29). If the template is RNA, the starting structure can be synthesized in the same way as when the template is DNA by adding a reverse transcription enzyme to the reaction solution composition, and then promoting amplification (RT-LAMP method). In the LAMP method, sufficient amplification product can be obtained in about 30 minutes. Consequently, if the objective is, for example, to rapidly determine the course of treatment, the LAMP method can be applied to the diagnosis of lymph node metastatic cancer in order to shorten the time required to detect nucleic acid. Because the result can be obtained quickly, it may be expected that the technique will be applied to intraoperative diagnosis.

There have already been reports on RT-PCR primers or probes to detect human CEA (Clinical Cancer Research, November 2000, Vol. 6, pp. 4176-4185). Nonetheless, there have been no report on primers applicable to the LAMP method, and it is desirable to develop them. Even for primers used in other nucleic acid amplification techniques, it would be preferable to have new primers in addition to the well-known primers, or to construct a primer set useful for measurements.

### BRIEF SUMMARY OF THE INVENTION

The present invention provides a novel primer for nucleic acid amplification in order to detect CEA.

As a result of extensive research in order to solve the above-mentioned problems, the present inventors realized the present invention in creating a primer for nucleic acid amplification which can detect CEA, and is effectively applicable to the LAMP method.

More specifically, the present inventions are as follows.

1. A primer to amplify nucleic acid and to detect CEA, wherein the primer comprising an oligonucleotide having a sequence selected from the group consisting of:

1) oligonucleotides with at least 5 or more continuous base from SEQ ID No.1 and/or its complementary strand, the oligonucleotides being selected from the 1900th to 2200th regions of a base sequence set out in SEQ ID No. 1, and regions complementary thereto;

2) oligonucleotides comprising a base sequence having one of SEQ ID Nos. 2 to 24;

3) complementary strands of the oligonucleotides described in the aforementioned 1) or 2);

4) oligonucleotides that can hybridize under stringent conditions with oligonucleotides described in any of aforementioned 1) to 3); and

5) oligonucleotides having a primer function selected from among the oligonucleotides described in aforementioned 1) to 4) that include a variant base sequence that has one to several bases substituted, deleted, inserted or added;

2. A primer to amplify nucleic acid and to detect CEA, wherein the primer comprising an oligonucleotide having a sequence selected from base sequences set out in SEQ ID Nos. 11 to 24;

3. The primer according to the aforementioned 1, wherein the nucleic acid is amplified by a LAMP method;

4. The primer according to the aforementioned 2, wherein the nucleic acid is amplified by a LAMP method;

5. A set of primer to amplify nucleic acid and to detect CEA, comprising at least two different kinds of primers comprising an oligonucleotide having a sequence selected from the group consisting of:

1) oligonucleotides with at least 5 or more continuous bases from SEQ ID No. 1 and/or its complementary strand, the oligonucleotides are selected from the 1900th to 2200th regions in a base sequence set out in SEQ ID No. 1, and regions complementary thereto;

2) oligonucleotides comprising a base sequence having one of SEQ ID Nos. 2 to 24;

3) complementary strands of the oligonucleotides described in the aforementioned 1) or 2);

4) oligonucleotides that can hybridize under stringent conditions with oligonucleotides described in any of aforementioned 1) to 3); and

5) oligonucleotides having a primer function selected from among the oligonucleotides described in aforementioned 1) to 4) that include a variant base sequence that has one to several bases substituted, deleted, inserted or added;

6. The set of primer according to aforementioned 5, wherein nucleic acid is amplified by a LAMP method;

7. The set of primer according to aforementioned 6, wherein at least four different kinds of primers are selected;

8. The set of primer according to aforementioned 6, wherein one of the primer recognizes at least six regions of the base sequence set out in SEQ ID No. 1 and/or a complementary strand thereto.

9. A set of primer selected from primers comprising an oligonucleotide of base sequences set out in any of SEQ ID Nos. 18 to 24, wherein one of the primer is selected from any of (a) SEQ ID Nos. 18 to 21, and the other is selected from any of (b) SEQ ID Nos. 22 to 24.

10. The set of according to aforementioned 9, further comprising a combination of primers selected from primers comprising sequences set out in SEQ ID Nos. 11 and/or 12.

11. The set of primer according to aforementioned 9, comprising primers selected from primers comprising an oligonucleotide having base sequences set out in any of SEQ ID Nos. 13 to 17, wherein one of the primers is selected from any of (e) SEQ ID Nos. 13 to 16, and the other is selected from any of (f) SEQ ID No. 17.

12. A set of primer to amplify nucleic acid and to detect CEA, the set comprising any one of:

1) a primer set comprising, as a primer, an oligonucleotide of base sequences set out in SEQ ID Nos. 11, 12, 18, and 22;

2) a primer set comprising, as a primer, an oligonucleotide of base sequences set out in SEQ ID Nos. 11, 12, 18, and 23;

3) a primer set comprising, as a primer, an oligonucleotide of base sequences set out in SEQ ID Nos. 11, 12, 18, and 24;

4) a primer set comprising, as a primer, an oligonucleotide of base sequences set out in SEQ ID Nos. 11, 12, 19, and 23;

5) a primer set comprising, as a primer, an oligonucleotide of base sequences set out in SEQ ID Nos. 11, 12, 19, and 24;

6) a primer set comprising, as a primer, an oligonucleotide of base sequences set out in SEQ ID Nos. 11, 12, 20, and 22;

7) a primer set comprising, as a primer, an oligonucleotide of base sequences set out in SEQ ID Nos. 11, 12, 20, and 23;

8) a primer set comprising, as a primer, an oligonucleotide of base sequences set out in SEQ ID Nos. 11, 12, 20, and 24;

9) a primer set comprising, as a primer, an oligonucleotide of base sequences set out in SEQ ID Nos. 11, 12, 21, and 22;

10) a primer set comprising, as a primer, an oligonucleotide of base sequences set out in SEQ ID Nos. 11, 12, 21, and 23; and

11) a primer set comprising, as a primer, an oligonucleotide of base sequences set out in SEQ ID Nos. 11, 12, 21, and 24.

13. A set of primer to amplify nucleic acid and to detect CEA, the set comprising any one of:

1) a primer set comprising, as a primer, an oligonucleotide of base sequences set out in SEQ ID Nos. 11, 12, 13, 17, 18, and 22;

2) a primer set comprising, as a primer, an oligonucleotide of base sequences set out in SEQ ID Nos. 11, 12, 13, 17, 18, and 23;

3) a primer set comprising, as a primer, an oligonucleotide of base sequences set out in SEQ ID Nos. 11, 12, 13, 17, 18, and 24;

4) a primer set comprising, as a primer, an oligonucleotide of base sequences set out in SEQ ID Nos. 11, 12, 14, 17, 18, and 24;

5) a primer set comprising, as a primer, an oligonucleotide of base sequences set out in SEQ ID Nos. 11, 12, 15, 17, 18, and 24;

6) a primer set comprising, as a primer, an oligonucleotide of base sequences set out in SEQ ID Nos. 11, 12, 13, 17, 19, and 23;

7) a primer set comprising, as a primer, an oligonucleotide of base sequences set out in SEQ ID Nos. 11, 12, 13, 17, 19, and 24;

8) a primer set comprising, as a primer, an oligonucleotide of base sequences set out in SEQ ID Nos. 11, 12, 13, 17, 20, and 22;

9) a primer set comprising, as a primer, an oligonucleotide of base sequences set out in SEQ ID Nos. 11, 12, 14, 17, 20, and 22;

10) a primer set comprising, as a primer, an oligonucleotide of base sequences set out in SEQ ID Nos. 11, 12, 15, 17, 20, and 22;

11) a primer set comprising, as a primer, an oligonucleotide of base sequences set out in SEQ ID Nos. 11, 12, 13, 17, 20, and 23;

12) a primer set comprising, as a primer, an oligonucleotide of base sequences set out in SEQ ID Nos. 11, 12, 13, 17, 20, and 24;

13) a primer set comprising, as a primer, an oligonucleotide of base sequences set out in SEQ ID Nos. 11, 12, 14, 17, 20, and 24;

14) a primer set comprising, as a primer, an oligonucleotide of base sequences set out in SEQ ID Nos. 11, 12, 15, 17, 20, and 24;

15) a primer set comprising, as a primer, an oligonucleotide of base sequences set out in SEQ ID Nos. 11, 12, 16, 17, 20, and 24;

16) a primer set comprising, as a primer, an oligonucleotide of base sequences set out in SEQ ID Nos. 11, 12, 13, 17, 21, and 22;

17) a primer set comprising, as a primer, an oligonucleotide of base sequences set out in SEQ ID Nos. 11, 12, 13, 17, 21, and 23;

18) a primer set comprising, as a primer, an oligonucleotide of base sequences set out in SEQ ID Nos. 11, 12, 14, 17, 21, and 23;

19) a primer set comprising, as a primer, an oligonucleotide of base sequences set out in SEQ ID Nos. 11, 12, 15, 17, 21, and 23;

20) a primer set comprising, as a primer, an oligonucleotide of base sequences set out in SEQ ID Nos. 11, 12, 16, 17, 21, and 23;

21) a primer set comprising, as a primer, an oligonucleotide of base sequences set out in SEQ ID Nos. 11, 12, 16, 17, 21, and 24;

22) a primer set comprising, as a primer, an oligonucleotide of base sequences set out in SEQ ID Nos. 11, 12, 13, 17, 21, and 24;

23) a primer set comprising, as a primer, an oligonucleotide of base sequences set out in SEQ ID Nos. 11, 12, 14, 17, 21, and 24; and

24) a primer set comprising, as a primer, an oligonucleotide of base sequences having SEQ ID Nos. 11, 12, 15, 17, 21, and 24;

14. A method for detecting nucleic acid using the primer according to the aforementioned 1;

15. A method for detecting nucleic acid using the primer according to the aforementioned 2;

16. A method for detecting nucleic acid using the set of primer according to the aforementioned 4;

17. A method for detecting nucleic acid using the set of primer according to the aforementioned 8;

18. A method for detecting nucleic acid using the set of primer according to the aforementioned 11;

19. A method for detecting nucleic acid using the set of primer according to the aforementioned 12.

### BRIEF DESCRIPTION OF THE DRAWINGS

FIG. 1 is a drawing showing the change of fluorescence intensity against the reaction time in the case of using primer set No.22.

FIG. 2 is a drawing showing the change of fluorescence intensity against the reaction time in the case of using primer set No.23.

FIG. 3 is a drawing showing the change of fluorescence intensity against the reaction time in the case of using primer set No.24.

### DETAILED DESCRIPTION OF THE INVENTION

Design of Primer

The present invention offers a primer for nucleic acid amplification that can be used in a method to amplify the nucleic acid of human CEA, preferably, in the LAMP method. Said primer is designed by selecting a suitable oligonucleotide including at least 5 or more continuous bases from the base sequence, and/or its complementary sequence, having the sequence set out in SEQ ID No. 1. The base sequence described in SEQ ID No. 1 is based on Genbank accession No. M17303.

The basic approach to the primers to be used in the LAMP method is like that described in U.S. Pat. No.6,410,278. Specifically, the regions F3c, F2c and F1c in order from the 3′ end side of the target DNA to be amplified, and the regions R3, R2 and R1 from 5′ end side are stipulated. Oligonucleotide strands that include substantially the same base sequences or a substantially complementary base sequences are selected in relation to at least these 6 regions, and at least 4 types of primers are designed.

“Substantially the same base sequence” is defined as follows. Specifically, when a complementary sequence, which has been synthesized using a given sequence as a template, is hybridized in relation to the target base sequence and provides the starting point for complementary strand synthesis, this sequence is substantially the same as the target base sequence. For example, substantially the same base sequence in relation to F2 includes not only the base sequence that is completely the same as F2, but also abase sequence that functions as a template to provide a base sequence that can be the starting point for complementary strand synthesis by hybridizing to F2.

The terms the “same” or “complementary” used in order to characterize the base sequence comprising the oligonucleotides based on the present invention do not have to mean completely the same or perfectly complementary. Specifically, the “same as a given sequence” can include a complementary sequence to the base sequence that can be hybridized in relation to a given sequence. On the other hand, “complementary” means that hybridization is possible under stringent conditions, and that a 3′ end is provided that is the starting point for complementary strand synthesis.

The primer in the present invention has enough strand length to be able to base bind with the complementary strand while maintaining the necessary specificity in the environments provided in the various types of nucleic acid synthesis reactions described below. Specifically, this is 5 to 200 bases, and preferably, 10 to 50 bases. Because the well-known polymerase that catalyzes the sequence-dependent nucleic acid synthesis reaction can identify a minimum primer strand length of around 5 bases, the strand length of the part to be hybridized must also be around 5 bases. Moreover, it is preferable to maintain a length of 10 bases or more in order to maintain specificity as a base sequence. On the other hand, a base sequence that is too long is difficult to prepare by chemical synthesis, and therefore the aforementioned strand length is indicated in an example of the preferable range.

The term “template” used in the present invention means the nucleic acid on the side that is the template for complementary strand synthesis. The complementary strand that has a base sequence complementary to the template means a strand that can be hybridized by the template, but ultimately the relationship of the two is simply relative. Specifically, a strand synthesized as a complementary strand can function again as a template. That is, a complementary strand can be a template.

In the present invention, the primer selected from the base sequence of the target DNA comprises the FIP (forward inner primer), F3 primer (forward outer primer), RIP (reverse inner primer) or R3 primer (reverse outer primer).

The FIP is designed to have on the 3′ end a base sequence of the F2 region substantially complementary with the F2c region of the target DNA, and to have on the 5′ end a base sequence substantially the same as the F1c region of the target DNA. In this case, a sequence not dependent on the target DNA may mediate between the F2 and F1c sequences. The permissible length of this sequence not dependent on the target DNA may be 0 to 50 bases, preferably, 0 to 40 bases.

F3 primer is designed to have substantially the same sequence as the F3 region substantially complementary with the F3c region of the target DNA.

RIP is designed to have on the 3′ end a base sequence of the R2 region substantially complementary with the R2c region of the target DNA, and to have on the 5′ end a base sequence substantially the same as the R1c region of the target DNA. As the same with FIP, the RIP also may have a sequence not dependent on the target DNA mediating between the R2 and R1c sequences.

R3 primer is designed to have substantially the same sequence as the R3 region substantially complementary with the R3c region of the target DNA.

In the LAMP method, it is possible to shorten the amplification time by sharing at least one or more type of loop primer (EP 1327679 A1). A loop primer is a one-strand part that loops on the 5′ end in a dumbbell structure, specifically, for example, a loop primer is a primer that has a complementary sequence between the R1 and R2 regions, or between the F1 and F2 regions. The starting points of DNA synthesis can be increased using a loop primer. This loop primer is designed so that the FIP or RIP produced in the DNA synthesis process hybridizes to a loop region that is not hybridized.

The primer of the present invention is designed by selecting regions according the aforementioned principles. The structural genes of human CEA are configured by the DNA strand and its complement that is comprised of the 2541 bases indicated in SEQ ID No. 1.

In careful consideration of the primer region accounting to the present invention such as the base composition, GC (Guanine and Cytosine) content, secondary structure, and Tm (melting temperature) value, etc., the length of the base sequence that recognizes the DNA region may be selected from a sequence of at least 5 bases or more, preferably from 10 to 30 bases, and more preferably, from 17 to 25 bases. The Tm value can generally be derived by the nearest neighbor method. The Tm value of the DNA region may be selected from about 55 to 65° C., preferably about 58 to 64° C.; and the GC content may be selected from about 40 to 70%, preferably about 50 to 65%.

Under these conditions, the primer region selected in the present invention includes base sequences from the 1900th to 2200th base in the sequence set out in SEQ ID No. 1, and in the region of the complementary strand thereof.

The primer of the present invention in designed by selecting from: 1) oligonucleotides with at least 5 or more bases, included in the region of the base sequence from the 1900th to 2200th base of the base sequence set out in SEQ ID No. 1, and/or in the complementary strand thereof; 2) oligonucleotides comprising the base sequence set out in SEQ ID Nos. 11 to 24; 3) the complementary strands of the oligonucleotides described in the aforementioned 1) or 2); 4) oligonucleotides that can hybridize under stringent conditions with oligonucleotides described in any of aforementioned 1) to 3); and 5) oligonucleotides having a primer function selected from among the oligonucleotides described in aforementioned 1) to 4) that include a variant base sequence that has one to several bases substituted, deleted, inserted or added.

The oligonucleotides may be produced by well-known techniques in the art, for example, they may be synthesized chemically. Or, natural nucleic acids may be cut by restricting enzymes, etc., and altered or connected into the structures of the aforementioned base sequences. Specifically, synthesis is possible using oligonucleotide synthesizer (Expedite Model 8909 DNA Synthesizer, manufactured by Applied Biosystems Co.), etc. It is also possible to use well-known manufacturing methods for the synthesis of oligonucleotides that substitute, delete, insert or add one or several bases. For example, site-specific variant incorporation, homologous recombination, primer extension or PCR may be used singly or suitably combined. The methods described in, for example, Molecular Cloning: A Laboratory Manual, Ed. 2, Sambrook, et al., ed., Cold Spring Harbor Laboratory, Cold Spring Harbor, New York, 1989; “Genetic Engineering Lab Manual”, M. Muramatsu, Maruzen Co., Ltd., 1988; “PCR Technology, Principles and Applications of DNA Amplification”, Ehrlich, HE. ed., Stockton Press, 1989, or variants of these may be employed, and technology such as that of Ulmer (Science (1983) 219; 666) may be utilized.

Stringent hybridization conditions may be selected from those well known in the art. As one example, after hybridizing for one night at 420° C. in a solution containing 50% formamide, 5×SSC (150 mM NaCl, 15 mM trisodium citrate), 50 mM sodium phosphate, pH 7.6, 5×Denhart's solution, 10% dextran sulfate and 20 μg/mL of DNA, a primary is washed in 2×SSC-0.1% SDS at room temperature followed by a secondary wash with 0.1×SSC-0.1% SDS at approximately 65° C.

Because the template of the nucleic acid to be amplified of the present invention is the mRNA of CEA, the primer used must be designed so that the genomic DNA included in the specimen is not amplified. Specifically, it is preferable that at least one of the primers included in the primer set of the present invention contain a region that straddles multiple exons in the human CEA gene. By employing this technique, it is possible to exclude amplification of sequences derived from genomic DNA, and to selectively amplify sequences derived from the mRNA of human CEA.

Primer Set

When amplifying nucleic acid using the primers of the present invention, at least 2 primers are combined into a primer set. In the LAMP method, 4 types of primers (FIP, F3 primer, RIP, and R3 primer) are combined into a primer set. Further, one or more kinds of loop primer may also be combined in a primer set.

RT-LAMP Method

The RT-LAMP method is the same as the LAMP method when an RNA template is used, and the basic approach is like that described in U.S. Pat. No. 6,410,278. In the RT-LAMP method, the starting structure of the LAMP method is synthesized while synthesizing the cDNA from the template RNA. Specifically, the target DNA is amplified by passing through the following Step 1), and then repeatedly growing DNA by repeating Steps 2) to 5).

1) FIP binds to the template RNA strand, and the complementary DNA strand is grown on the template RNA strand. This reaction uses a reverse transcription enzyme, for example, a reverse transcription enzyme derived from AMV (Avian Myeloblastosis Virus).

2) As the F3 primer displaces the DNA strand synthesized in above 1) from the FIP of the RNA template, a complementary DNA strand is grown on the template RNA strand. The growth of subsequent DNA strands is based on DNA polymerase.

3) RIP binds with the DNA strand displaced in above 2), and a DNA strand is grown.

4) As the R3 primer displaces the DNA strand grown in above 3) from the RIP, a complementary DNA strand is grown on the DNA strand from the FIP, and the starting structure for the LAMP method is synthesized.

5) Because the both ends of the DNA strand displaced in above 4) have complementary sequences within the sequence of the same DNA strand, they respectively hybridize to form loop structures on both ends.

For example, if an enzyme like BcaDNA polymerase is used, which has both a reverse transcriptase activity and a DNA polymerase activity, the aforementioned reaction can be conducted with one enzyme.

Measurement Method

In the LAMP method, because the synthesized DNA strand has a complementary strand corresponding to its original strand, the majority of it forms a bond to a base. It is possible to detect amplification products using this characteristic. If nucleic acid amplification using the primer of the present invention is conducted in the presence of a fluorescent dye that is a double-stranded intercalator such as ethidium bromide, Syber Green I or Pico Green, the increase in the intensity of fluorescence can be observed as the product increase. If this is monitored, it is possible to simultaneously follow the amplification of DNA and the increase of fluorescence in a closed system (hereinafter simply referred to “real time method”, see, Summary of Clinical Test Methods, Ed. 31, page 1318; Japanese Patent Provisional Publication No. 2001-242169).

Reagents, Reagent Kit, Other

The various reagents necessary when detecting nucleic acid using the primer of the present invention may be made into a pre-packaged kit. Specifically, this may be a kit comprising: the oligonucleotides necessary for the primers or substitution primers of the complementary strand synthesis of the present invention; the enzymes having reverse transcription activity; dNTP, which is the substrate for complementary strand synthesis; DNA polymerase to conduct complementary strand synthesis of the strand substitution model; buffer solution to provide suitable conditions for the enzyme reaction, and the reagents necessary in order to detect the reaction products.

The present invention applies to the primers and primer set for nucleic acid amplification, as well as to the nucleic acid detection method using these primers, the detection reagents used in this nucleic acid,detection method, the nucleic acid detection kit, and the entire system of nucleic acid detection.

### EXAMPLE

The present invention is illustrated in the following Example, but is not limited to these examples.

Selection of Region

With respect to the base sequence for human CEA set out in SEQ ID No. 1, the position of a suitable range in the LAMP method was studied using probe design software. As a result thereof, a Tm value of 58.5 to 63.5° C. for the region of F1c and R1c, a Tm value of 61.5 to 62.5° C. for F2 and R2, and a Tm value of 58.5 to 62.5° C. for F3 and R3, the following particulars were selected. The selected regions are included in the region of Nos. 1900 to 2200 of the base sequence set out in SEQ ID No. 1 and its complementary strand.

F1c: Region of the strand complementary to the base position in the sequence set out in SEQ ID No. 1:

Nos. 2054-2034 (SEQ ID No. 2)

F2: Region of the base position on the sequence set out in SEQ ID No. 1:

Nos. 1951 to 1966 (SEQ ID No. 3)

Nos. 1952 to 1968 (SEQ ID No. 4)

Nos. 1950 to 1966 (SEQ ID No. 5)

Nos. 1957 to 1973 (SEQ ID No. 6)

R1c: Region of the base position on the sequence set out in SEQ ID No. 1:

Nos. 2062 to 2084 (SEQ ID No. 7)

R2: Region of the strand complementary to the base position on the sequence set out in SEQ ID No. 1:

Nos. 2134 to 2114 (SEQ ID No. 8)

Nos. 2133 to 2114 (SEQ ID No. 9)

Nos. 2138 to 2120 (SEQ ID No. 10)

F3: Region of the base position on the sequence set out in SEQ ID No. 1:

Nos. 1906 to 1922 (SEQ ID No. 11)

R3: Region of the base position on the sequence set out in SEQ ID No. 1:

Nos. 2189 to 2172 (SEQ ID No. 12)

loop F: Region of the strand complementary to the base position on the sequence set out in SEQ ID No. 1:

Nos. 1997 to 1978 (SEQ ID No. 13)

Nos. 1994 to 1975 (SEQ ID No. 14)

Nos. 1996 to 1978 (SEQ ID No. 15)

Nos. 1995 to 1976 (SEQ ID No. 16)

loop R: Region of the base position on the sequence set out in SEQ ID No. 1:

Nos. 2086 to 2109 (SEQ ID No. 17)

Primer Design

The following nucleic acid amplification primers for use in the LAMP method were obtained from the sequences of the selected regions.

FIP: Primer comprising the base sequence connecting Regions F1c and F2

AFA I: (SEQ ID No. 18) Connecting the sequences set out in SEQ ID Nos. 2 and 3

AFA II: (SEQ ID No. 19) Connecting the sequences set out in SEQ ID Nos. 2 and 4

AFA III: (SEQ ID No. 20) Connecting the sequences set out in SEQ ID Nos. 2 and 5

AFA V: (SEQ ID No. 21) Connecting the sequences set out in SEQ ID Nos. 2 and 6

RIP: Primer comprising the base sequence connecting Regions R1c and R2

ARA I: (SEQ ID No. 22) Connecting the sequences set out in SEQ ID Nos. 7 and 8

ARA II: (SEQ ID No. 23) Connecting the sequences set out in SEQ ID Nos. 7 and 9

ARA V: (SEQ ID No. 24) Connecting the sequences set out in SEQ ID Nos. 7 and 10

F3 primer: Primer comprising the base sequence identified with the various sequence numbers.

AF3: (SEQ ID No. 11)

R3 primer: Primer comprising the base sequence identified with the various sequence numbers.

RF3: (SEQ ID No. 12)

Loop primer F: Primer comprising the base sequence identified with the various sequence numbers.

LPF: (SEQ ID No. 13)

LPF II: (SEQ ID No. 14)

LPF III: (SEQ ID No. 15)

LPF IV: (SEQ ID No. 16)

Loop primer R: Primer comprising the base sequence identified with the various sequence numbers.

LPR: (SEQ ID No. 17)

### EXPERIMENT

An experiment using the RT-LAMP method to measure the aforementioned types of primers in the following combinations was conducted for the purpose of investigating the time required by the various primer sets from the beginning of reaction until amplification could be confirmed.

1) Method of Preparing Human CEA RNA Samples Human CEA cDNA was prepared from total RNA derived from LS180 (colon cancer cells) by RT-PCR using a primer designed based on the human CEA base sequence. A transcript product was synthesized using an in vitro transcript system (Riboprobe in vitro transcription system (produced by Promega)) from human CEA cDNA cloned by pGEM-32 (produced by Promega) The RNA concentration of the source solution obtained was calculated using absorbency measurements at 260 nm, and based on that value dilutions were prepared with 50 ng/mL yeast RNA (produced by Amibon) to make templates with the number of human CEA RNA copies at 60,000, 6,000, 600, 60, 6, and 0 (control).

2) Primer Set

The various types of primers were combined (Table 1). The numbers in the table indicate the SEQ ID No., and indicate the fact that the primers comprising the oligonucleotides of the base sequence identified with the various sequence numbers were used in the set.

TABLE 1

| Primer | | | F3 | R3 | Loop | Loop |
|---|---|---|---|---|---|---|
| Set No. | FIP | RIP | Primer | Primer | Primer F | Primer R |
| 1 | 18 | 22 | 11 | 12 | 13 | 17 |
| 2 | 18 | 23 | 11 | 12 | 13 | 17 |
| 3 | 18 | 24 | 11 | 12 | 13 | 17 |
| 4 | 18 | 24 | 11 | 12 | 14 | 17 |
| 5 | 18 | 24 | 11 | 12 | 15 | 17 |
| 6 | 19 | 23 | 11 | 12 | 13 | 17 |
| 7 | 19 | 24 | 11 | 12 | 13 | 17 |
| 8 | 20 | 22 | 11 | 12 | 13 | 17 |
| 9 | 20 | 22 | 11 | 12 | 14 | 17 |
| 10 | 20 | 22 | 11 | 12 | 15 | 17 |
| 11 | 20 | 23 | 11 | 12 | 13 | 17 |
| 12 | 20 | 24 | 11 | 12 | 13 | 17 |
| 13 | 20 | 24 | 11 | 12 | 14 | 17 |
| 14 | 20 | 24 | 11 | 12 | 15 | 17 |
| 15 | 20 | 24 | 11 | 12 | 16 | 17 |
| 16 | 21 | 22 | 11 | 12 | 13 | 17 |
| 17 | 21 | 23 | 11 | 12 | 13 | 17 |
| 18 | 21 | 23 | 11 | 12 | 14 | 17 |
| 19 | 21 | 23 | 11 | 12 | 15 | 17 |
| 20 | 21 | 23 | 11 | 12 | 16 | 17 |
| 21 | 21 | 24 | 11 | 12 | 16 | 17 |
| 22 | 21 | 24 | 11 | 12 | 13 | 17 |
| 23 | 21 | 24 | 11 | 12 | 14 | 17 |
| 24 | 21 | 24 | 11 | 12 | 15 | 17 |

3) Reaction Solution Composition (in 23 μL of Reaction Mixture)

| | | |
|---|---|---|
| dNTPs (GIBCO) | 0.4 | mM |
| MgSO₄ | 2 | mM |
| dithiothreitol | 5 | mM |
| betaine (Sigma) | 640 | mM |

Thermopol Buffer (New England BioLabs)

| | | |
|---|---|---|
| AMV reverse transcription enzyme (Promega) | 0.05 | U |
| Bst DNA polymerase (New England BioLabs) | 0.64 | U |
| ethidium bromide | 0.25 | μg/mL |

Primers

| | |
|---|---|
| FIP | 40 pmol |
| RIP | 40 pmol |
| F3 primer | 5 pmol |
| R3 primer | 5 pmol |
| Loop primer F | 20 pmol |
| Loop primer R | 20 pmol |

4) RT-LAMP Method

2 μL of human CEA RNA sample was added to 23 μL of reaction solution containing the aforementioned 6 types of primers, and this was heated for 1 hour at 65° C.

5) Confirmation of Amplification

Because the amplification product has a double strand structure, fluorescence was induced by intercalating ethidium bromide. The increase of the intensity of fluorescence was measured by real time method using PRISM 7700 manufactured by ABI.

6) Result

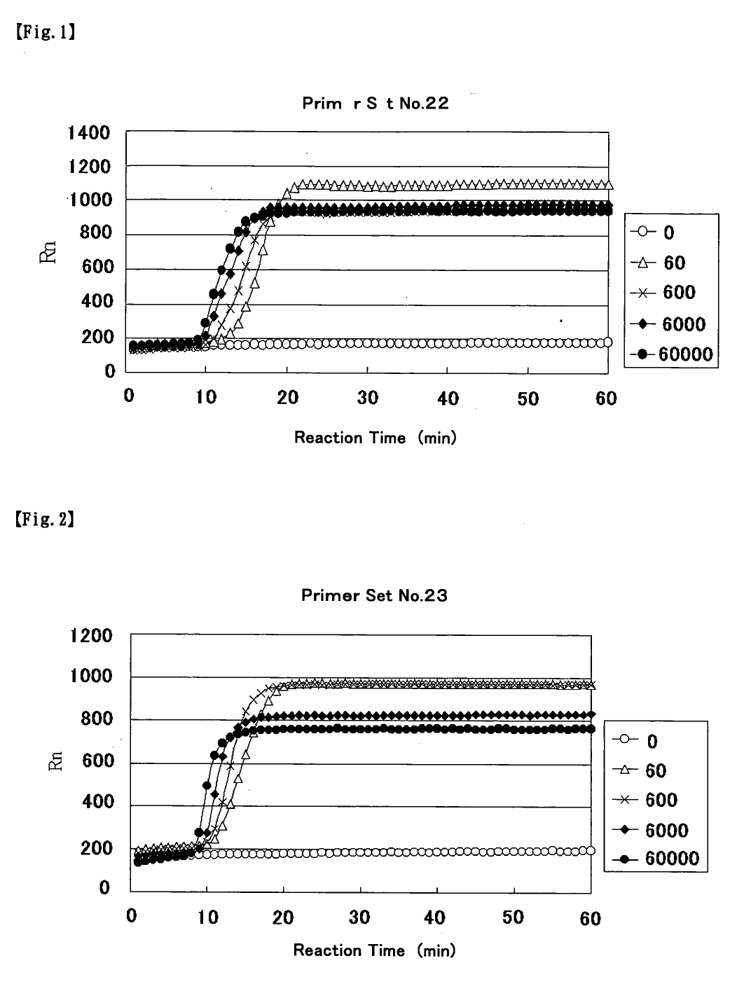

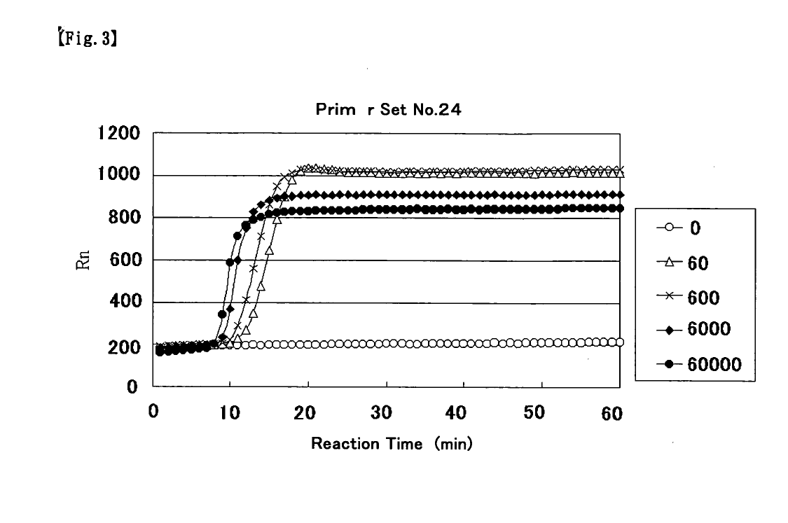

FIGS. 1 to 3 show results obtained using the primer sets of 22 to 24. In each of the primer sets, it was confirmed that it took shorter time to amplify the template of the sample to be measured that contains more amount of template of CEA. In the primer sets 1 to 3, amplification was confirmed within 20 minutes in the case of 60 copies, while amplification was confirmed in about 10 minutes in the case of 6000 copies.

What is claimed is:

1. A primer to amplify nucleic acid and to detect carcinoembryonic antigen (CEA), wherein the primer comprising an oligonucleotide having a sequence selected from the group consisting of:
1) oligonucleotides with at least 5 or more continuous bases from SEQ ID No. 1 and/or its complementary-strand, the oligonucleotides being selected from the 1900th to 2200th regions of a base sequence having SEQ ID No. 1, and regions complementary thereto;
2) oligonucleotides comprising a base sequence having one of SEQ ID Nos. 2 to 24;
3) complementary strands of the oligonucleotides described in the aforementioned 1) or 2);
4) oligonucleotides that can hybridize under stringent conditions with oligonucleotides described in any of aforementioned 1) to 3); and
5) oligonucleotides having a primer function selected from among the oligonucleotides described in aforementioned 1) to 4) that include a variant base sequence that has one to several bases substituted, deleted, inserted or added.

2. A primer to amplify nucleic acid and to detect CEA, wherein the primer comprising an oligonucleotide having a sequence selected from base sequences having SEQ ID Nos. 11 to 24.

3. The primer according to claim 1, wherein the nucleic acid is amplified by a LAMP method.

4. The primer according to claim 2, wherein the nucleic acid is amplified by a LAMP method.

5. A set of primer to amplify nucleic acid and to detect CEA, comprising at least two different kinds of primers comprising an oligonucleotide from having a sequence selected from the group consisting of:
1) oligonucleotides with at least 5 or more continuous bases from SEQ ID No. 1 and/or its complementary strand, the oligonucleotides being selected from the 1900th to 2200th regions of a base sequence set out in SEQ ID No. 1, and regions complementary thereto;
2) oligonucleotides comprising a base sequence having one of SEQ ID Nos. 2 to 24;
3) complementary strands of the oligonucleotides described in the aforementioned 1) or 2);
4) oligonucleotides that can hybridize under stringent conditions with oligonucleotides described in any of aforementioned 1) to 3); and
5) oligonucleotides comprising a primer function selected from among the oligonucleotides described in aforementioned 1) to 4) that include a variant base sequence that has one to several bases substituted, deleted, inserted or added.

6. The set of primer according to claim 5, wherein nucleic acid is amplified by a LAMP method.

7. The set of primer according to claim 6, wherein at least four different kinds of primers are selected.

8. The set of primer according to claim 6, wherein one of the primer recognizes at least six regions of the base sequence set out in SEQ ID No. 1 and/or a complementary strand thereto.

9. A set of primer selected from primers comprising an oligonucleotide of base sequences set out in any of SEQ ID Nos. 18 to 24, wherein one of the primer is selected from any of(a) SEQ ID Nos. 18 to 20, and the other is selected from any of (b) SEQ ID Nos. 22 to 24.

10. The set of primer according to claim 9, further comprising a combination of primers selected from primers comprising sequences set out in SEQ ID Nos. 11 and/or 12.

11. The set of primer according to claim 9, comprising primers selected from primers comprising an oligonucleotide having base sequences set out in any of SEQ ID Nos. 13 to 17, wherein one of the primers is selected from any of (e) SEQ ID Nos. 13 to 16, and the other is selected from any of (f) SEQ ID No. 17.

12. A set of primer to amplify nucleic acid and to detect CEA, the set comprising any one of:
1) a primer set comprising, as a primer, an oligonucleotide of base sequences set out in SEQ ID Nos. 11, 12, 18, and 22;
2) a primer set comprising, as a primer, an oligonucleotide of base sequences set out in SEQ ID Nos. 11, 12, 18, and 23;
3) a primer set comprising, as a primer, an oligonucleotide of base sequences set out in SEQ ID Nos. 11, 12, 18, and 24;
4) a primer set comprising, as a primer, an oligonucleotide of base sequences set out in SEQ ID Nos. 11, 12, 19, and 23;
5) a primer set comprising, as a primer, an oligonucleotide of base sequences set out in SEQ ID Nos. 11, 12, 19, and 24;
6) a primer set comprising, as a primer, an oligonucleotide of base sequences set out in SEQ ID Nos. 11, 12, 20, and 22;
7) a primer set comprising, as a primer, an oligonucleotide of base sequences set out in SEQ ID Nos. 11, 12, 20, and 23;
8) a primer set comprising, as a primer, an oligonucleotide of base sequences set out in SEQ ID Nos. 11, 12, 20, and 24;
9) a primer set comprising, as a primer, an oligonucleotide of base sequences set out in SEQ ID Nos. 11, 12, 21, and 22;
10) a primer set comprising, as a primer, an oligonucleotide of base sequences set out in SEQ.ID Nos. 11, 12, 21, and 23; and
11) a primer set comprising, as a primer, an oligonucleotide of base sequences set out in SEQ ID Nos. 11, 12, 21, and 24.

13. A set of primer to amplify nucleic acid and to detect CEA, the set comprising any one of:
1) a primer set comprising, as a primer, an oligonucleotide of base sequences set out in SEQ ID Nos. 11, 12, 13, 17, 18, and 22;
2) a primer set comprising, as a primer, an oligonucleotide of base sequences set out in SEQ ID Nos. 11, 12, 13, 17, 18, and 23;
3) a-primer set comprising, as a primer, an oligonucleotide of base sequences set out in SEQ ID Nos. 11, 12, 13, 17, 18, and 24;
4) a primer set comprising, as a primer, an oligonucleotide of base sequences set out in SEQ ID Nos. 11, 12, 14, 17, 18, and 24;
5) a primer set comprising, as a primer, an oligonucleotide of base sequences set out in SEQ ID Nos. 11, 12, 15, 17, 18, and 24;
6) a primer set comprising, as a primer, an oligonucleotide of base sequences set out in SEQ ID Nos. 11, 12, 13, 17, 19, and 23;
7) a primer set comprising, as a primer, an oligonucleotide of base sequences set out in SEQ ID Nos. 11, 12, 13, 17, 19, and 24;
8) a primer set comprising, as a primer, an oligonucleotide of base sequences set out in SEQ ID Nos. 11, 12, 13, 17, 20, and 22;
9) a primer set comprising, as a primer, an oligonucleotide of base sequences set out in SEQ ID Nos. 11, 12, 14, 17, 20, and 22;
10) a primer set comprising, as a primer, an oligonucleotide of base sequences set out in SEQ ID Nos. 11, 12, 15, 17, 20, and 22;
11) a primer set comprising, as a primer, an oligonucleotide of base sequences set out in SEQ ID Nos. 11, 12, 13, 17, 20, and 23;
12) a primer set comprising, as a primer, an oligonucleotide of base sequences set out in SEQ ID Nos. 11, 12, 13, 17, 20, and 24;
13) a primer set comprising, as a primer, an oligonucleotide of base sequences set out in SEQ ID Nos. 11, 12, 14, 17, 20, and 24;
14) a primer set comprising, as a primer, an oligonucleotide of base sequences set out in SEQ ID Nos. 11, 12, 15, 17, 20, and 24;
15) a primer set comprising, as a primer, an oligonucleotide of base sequences set out in SEQ ID Nos. 11, 12, 16, 17, 20, and 24;
16) a primer set comprising, as a primer, an oligonucleotide of base sequences set out in SEQ ID Nos. 11, 12, 13, 17, 21, and 22;
17) a primer set comprising, as a primer, an oligonucleotide of base sequences set out in SEQ ID Nos. 11, 12, 13, 17, 21, and 23;
18) a primer set comprising, as a primer, an oligonucleotide of base sequences set out in SEQ ID Nos. 11, 12, 14, 17, 21, and 23;
19) a primer set comprising, as a primer, an oligonucleotide of base sequences set out in SEQ ID Nos. 11, 12, 15, 17, 21, and 23;
20) a primer set comprising, as a primer, an oligonucleotide of base sequences set out in SEQ ID Nos. 11, 12, 16, 17, 21, and 23;
21) a primer set comprising, as a primer, an oligonucleotide of base sequences set out in SEQ ID Nos. 11, 12, 16, 17, 21, and 24;
22) a primer set comprising, as a primer, an oligonucleotide of base sequences set out in SEQ ID Nos. 11, 12, 13, 17, 21, and 24;
23) a primer set comprising, as a primer, an oligonucleotide of base sequences set out in SEQ ID Nos. 11, 12, 14, 17, 21, and 24; and
24) a primer set comprising, as a primer, an oligonucleotide of base sequences set out in SEQ ID Nos. 11, 12, 15, 17, 21, and 24.

14. A method for detecting nucleic acid using the primer according to claim 1.

15. A method for detecting nucleic acid using the primer according to claim 2.

16. A method for detecting nucleic acid using the set of primer according to claim 4.

17. A method for detecting nucleic acid using the set of primer according to claim 8.

18. A method for detecting nucleic acid using the set of primer according to claim 11.

19. A method for detecting nucleic acid using the set of primer according to claim 12.


SEQUENCE LISTING (24 sequences)

SEQ ID NO: 1 - DNA, 2541 bp, Homo sapiens
cgaccagcagaccagacagtcacagcagccttgacaaaacgttcctggaactcaagcacttctccacagaggaggacagagcagacagcagagaccatggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccatgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatagcagccctggtgtagtttcttcatttcaggaagactgacagttgttttgcttcttccttaaagcatttgcaacagctacagtctaaaattgcttctttaccaaggatatttacagaaaagactctgaccagagatcgagaccatcctagccaacatcgtgaaaccccatctctactaaaaatacaaaaatgagctgggcttggtggcgcgcacctgtagtcccagttactcgggaggctgaggcaggagaatcgcttgaacccgggaggtggagattgcagtgagcccagatcgcaccactgcactccagtctggcaacagagcaagactccatctc

SEQ ID NO: 2 - DNA, 21 bp, Artificial Sequence
  primer
taggtcccgttattatttggc

SEQ ID NO: 3 - DNA, 16 bp, Artificial Sequence
  primer
tctaacccatccccgc

SEQ ID NO: 4 - DNA, 17 bp, Artificial Sequence
  primer
ctaacccatccccgcag

SEQ ID NO: 5 - DNA, 17 bp, Artificial Sequence
  primer
ctctaacccatccccgc

SEQ ID NO: 6 - DNA, 17 bp, Artificial Sequence
  primer
ccatccccgcagtattc

SEQ ID NO: 7 - DNA, 23 bp, Artificial Sequence
  primer
tttgtctctaacttggctactgg

SEQ ID NO: 8 - DNA, 21 bp, Artificial Sequence
  primer
aagttccagatgcagagactg

SEQ ID NO: 9 - DNA, 20 bp, Artificial Sequence
  primer
agttccagatgcagagactg

SEQ ID NO: 10 - DNA, 19 bp, Artificial Sequence
  primer
ggagaagttccagatgcag

SEQ ID NO: 11 - DNA, 17 bp, Artificial Sequence
  primer
tcttacctttcgggagc

SEQ ID NO: 12 - DNA, 18 bp, Artificial Sequence
  primer
ccaaccagcactccaatc

SEQ ID NO: 13 - DNA, 20 bp, Artificial Sequence
  primer
tgcggtatcccattgatacg

SEQ ID NO: 14 - DNA, 20 bp, Artificial Sequence
  primer
ggtatcccattgatacgcca

SEQ ID NO: 15 - DNA, 19 bp, Artificial Sequence
  primer
gcggtatcccattgatacg

SEQ ID NO: 16 - DNA, 20 bp, Artificial Sequence
  primer
cggtatcccattgatacgcc

SEQ ID NO: 17 - DNA, 24 bp, Artificial Sequence
  primer
cgcaataattccatagtcaagagc

SEQ ID NO: 18 - DNA, 37 bp, Artificial Sequence
  primer
taggtcccgttattatttggctctaacccatccccgc

SEQ ID NO: 19 - DNA, 38 bp, Artificial Sequence
  primer
taggtcccgttattatttggcctaacccatccccgcag

SEQ ID NO: 20 - DNA, 38 bp, Artificial Sequence
  primer
taggtcccgttattatttggcctctaacccatccccgc

SEQ ID NO: 21 - DNA, 38 bp, Artificial Sequence
  primer
taggtcccgttattatttggcccatccccgcagtattc

SEQ ID NO: 22 - DNA, 44 bp, Artificial Sequence
  primer
tttgtctctaacttggctactggaagttccagatgcagagactg

SEQ ID NO: 23 - DNA, 43 bp, Artificial Sequence
  primer
tttgtctctaacttggctactggagttccagatgcagagactg

SEQ ID NO: 24 - DNA, 42 bp, Artificial Sequence
  primer
tttgtctctaacttggctactggggagaagttccagatgcag